(19) United States
(12) Patent Application Publication
(10) Pub. No.: US 2009/0042218 A1
(43) Pub. Date: Feb. 12, 2009

(54) Labeling enzyme

(76) Inventors: Kazunori Ikebukuro, Tokyo (JP); Koji Sode, Tokyo (JP); Akira Umezawa, Tokyo (JP); Kyoko Umezawa, Tokyo (JP); Hironobu Yamamoto, Saitama (JP)

Correspondence Address: NIXON & VANDERHYE, PC, 901 NORTH GLEBE ROAD, 11TH FLOOR, ARLINGTON, VA, 22203, US

(21) Appl. No.: 11/663,345

(22) PCT Filed: Sep. 21, 2005
(86) PCT No.: PCT/JP2005/017378

§ 371 Date: Feb. 6, 2008

(30) Foreign Application Priority Data
Sep. 22, 2004 (JP) 2004-275000

(51) Int. Cl. G01N 33/573; C12N 9/50; C12N 9/74

(52) U.S. Cl. 435/7.9; 435/219; 435/214

(57) ABSTRACT

The present invention relates to a labeling enzyme and a method of detecting and/or quantifying a target substance using this labeling enzyme. The present invention provides a labeling enzyme that catalyzes a reaction of gelling a substrate. By measuring changes in physical properties such as the film thickness and/or refractive index of a film produced by the gelling reaction catalyzed by the labeling enzyme of the present invention, it is possible to quickly and highly sensitively detect and/or quantify a target substance while minimizing the effects of coexisting substances.

### TECHNICAL FIELD

The present invention relates to a labeling enzyme and to a method of detecting and/or quantifying a target substance using the labeling enzyme.

### BACKGROUND ART

Currently, ELISA (enzyme-linked immunosorbent assay) and other forms of enzyme immunoassay (EIA) are widely used as methods for detecting and/or quantifying a target substance using the affinity between an antibody and an antigen, and are indispensable techniques in various kinds of diagnosis and biological testing. Biosensors called immunosensors having also been developed using enzyme immunoassay, and are widely used.

In ELISA measurement, an antigen or antibody (the target substance) is reacted with an antibody or antigen linked to a labeling enzyme, and the target substance is detected and/or quantified from the enzyme activity of the labeling enzyme. That is, in ELISA a signal obtained by molecular recognition of an antibody or antigen is amplified with an enzyme in order to achieve highly sensitive detection of a target substance.

Most of the enzymes used as labeling enzymes in ELISA are peroxidases (F. Patolsky et al., Langmuir, 15, 3703, 1999), alkaline phosphatases (E. Kim et al., Anal. Chem., 75, 5665, 2003), glucose oxidases (K. Kojima et al., Analytical Chemistry, 75(5), 1116, 2003) or galactosidases, and these labeling enzymes are also commonly used in immunosensors using ELISA (Kazunori Ikebukuro & Koji Sode, “Koso denkyoku hanno wo mochiita DNA hybridization no kokando-kenshutsu”, Electrochemistry, No. 72 Vol. 8, pp. 594 through 597, 2004). In these methods, reaction products generated by a reaction catalyzed by the labeling enzyme are generally detected by light absorption, fluorescence or light emission, and in the case of an immunosensor the enzyme reaction of the labeling enzyme is usually detected electrochemically. Other enzyme reaction detection methods include a method in which insoluble products are generated in a reaction by means of a labeling enzyme such as an alkaline phosphatase, and the sediment is detected using a quartz oscillator or other piezo element (Patolsky et al., Nat. Biotechnol., 19(3), 253, 2001).

### DISCLOSURE OF THE INVENTION

### Problems to be Solved by the Invention

The problem with conventional methods of detecting and/or quantifying target substances using an enzyme reaction is that they are vulnerable to the effects of impurities and coexisting substances, making it difficult to obtain stable measurement results. When insoluble products are generated using a labeling enzyme, moreover, they must be detected with a quartz oscillator, but the oscillation of a quartz oscillator is unstable in water, making highly sensitive detection difficult.

Under these circumstances, it is an object of the present invention to provide, in a method for measuring a target substance using an enzyme reaction, a method of detecting and/or quantifying a target substance easily and with high sensitivity while minimizing the effects of coexisting substances.

### Means for Solving the Problems

The inventors in this case perfected the present invention when they succeeded, as a result of exhaustive research aimed at solving these problems, in developing a method of detecting and/or quantifying a target substance easily and with high sensitivity while minimizing the effects of coexisting substances by measuring change in physical properties and particularly changes in the film thickness and/or refractive index of a gel film produced by a gelling reaction using an enzyme.

That is, the present invention relates to the following.

(1) A labeling enzyme that catalyzes a reaction of gelling a substrate.

(2) The labeling enzyme according to (1), wherein the labeling enzyme is a protease or peptidase.

(3) The labeling enzyme according to (1) or (2), wherein the labeling enzyme is an enzyme involved in a blood coagulation reaction.

(4) The labeling enzyme according to any one of (1) through (3), wherein the labeling enzyme is thrombin.

(5) The labeling enzyme according to any one of (1) through (4), wherein the substrate is a protein.

(6) The labeling enzyme according to any one of (1) through (5), wherein the substrate is fibrinogen.

(7) A method for detecting and/or quantifying a target substance, comprising:
(a) a step of reacting a target substance, a fixed molecule-recognizing element comprising a support having fixed thereon a molecule-recognizing element capable of specifically recognizing the target substance, and a labeled molecule-recognizing element capable of specifically recognizing the target substance and labeled with a labeling enzyme that catalyzes a reaction of gelling a substrate;
(b) a step of adding a substrate that is gelled by the catalytic action of the labeling enzyme to the fixed molecule-recognizing element/target substance/labeled molecule-recognizing element complex produced in step (a), thereby producing a gel on the support; and
(c) a step of measuring changes in the film thickness and/or refractive index of a film on the support comprising the gel produced in step (b).

(8) A method for detecting and/or quantifying a target substance, comprising:
(a) a step of reacting a target substance and a target substance labeled with a labeling enzyme that catalyzes a substrate-gelling reaction, with a fixed molecule-recognizing element comprising a support having fixed thereon a molecule-recognizing element capable of specifically recognizing the target substance;
(b) a step of adding a substrate that is gelled by the catalytic action of the labeling enzyme to the fixed molecule-recognizing element/target substance complex and the fixed molecule-recognizing element/labeled target substance produced in step (a), thereby producing a gel on the support; and
(c) a step of measuring changes in the film thickness and/or refractive index of a film on the support comprising the gel produced in step (b).

(9) The method for detecting and/or quantifying a target substance according to (7) or (8), wherein changes in the film thickness and/or refractive index of the film are measured by the interference enhanced reflection method or surface plasmon resonance method.

(10) A kit for measuring a target substance, comprising: a labeling enzyme that catalyzes a reaction of gelling a substrate; and a substrate that is gelled by the catalytic action of the labeling enzyme.

(11) A kit for measuring a target substance, comprising: a labeling enzyme that catalyzes a reaction of gelling a substrate; and a reagent for linking the labeling enzyme to a molecule-recognizing element capable of specifically recognizing a target substance.

(12) A biosensor for detecting and/or quantifying a target substance, comprising:
(a) a reaction part for reacting a first molecule-recognizing element fixed on a support and capable of specifically recognizing the target substance, a second molecule-recognizing element labeled with a labeling enzyme that catalyzes a reaction of gelling a substrate and capable of specifically recognizing the target substance, a substrate that is gelled by the catalytic action of the labeling enzyme, and the target substance; and
(b) a measurement part for measuring changes in the film thickness and/or refractive index of a film produced on the support by gelling caused by the aforementioned reaction.

(13) A biosensor for detecting and/or quantifying a target substance, comprising:
(a) a reaction part for reacting a target substance, a labeled substance labeled with a labeling enzyme that catalyzes a reaction of gelling a substrate, a fixed molecule-recognizing element fixed on a support and capable of specifically recognizing the target substance, and a substrate that is gelled by the catalytic action of the labeling enzyme; and
(b) a measurement part for measuring changes in the film thickness and/or refractive index of a film produced on the support by gelling caused by the aforementioned reaction.

### Labeling Enzyme and Substrate

A labeling enzyme that can be used in the present invention is an enzyme that can act on a substrate to produce a gel and thereby cause changes in the film thickness and/or refractive index of a film, without any particular limitation. Because a very small quantity of enzyme is required for a gelling reaction, a highly sensitive measurement system can be constructed with the present invention. The labeling enzyme of the present invention is linked to an antigen, antibody or other molecule-recognizing element, thereby labeling the molecule-recognizing element. Examples of labeling enzymes that can be used in the present invention include proteases, peptidases and the like, but are not limited to these. In this case, the substrate used in the present invention may be a protein, polypeptide, peptide or the like.

An enzyme involved in a blood coagulation reaction is preferred as the labeling enzyme used in the present invention. It is still more preferable to use thrombin as the labeling enzyme in the present invention, in which case the substrate is fibrinogen. This is because gelling of fibrinogen by thrombin is little affected by coexisting substances, allowing for more stable measurement. In this case, the fibrinogen substrate is hydrolyzed and gelled by thrombin, an enzyme involved in a blood coagulation reaction, producing insoluble fibrin. In this way, changes are produced in the film thickness and/or refractive index of a film.

In addition, a gelling reaction caused by an endotoxin can also be used favorably in the present reaction. Specifically, a horseshoe crab blood coagulation reaction can be used which is a gelling reaction caused by an endotoxin or glycan (β-D-glycan or the like) and a horseshoe crab blood cell extract (LAL, lysate). In particular, a gelling reaction caused by an endotoxin or glycan and a horseshoe crab blood cell extract is used as a limulus test in the fields of drug quality and other quality control, and is desirable as the gelling reaction of the present invention because it is easy to manage.

Looking more closely at the gelling caused by horseshoe crab blood components, the reaction proceeds as follows. A C factor system that reacts with and is activated by endotoxin and a G factor system that reacts with and is activated by β-D-glycan (a plant polysaccharide which is one component of fungal cell walls) have been confirmed in horseshoe crab lysate, and the pathways of these two systems are the same past a certain point. That is, a pro-clotting enzyme is activated to produce a clotting enzyme, which then partially hydrolyzes a coagulogen substrate, releasing peptide C and producing coagulin, resulting in a gel.

Reagents that are derived from horseshoe crab blood cell components and react specifically with endotoxin to produce a gel are sold by Wako Pure Chemical (Code No. 298-22341) and Seikagaku Corp. (Endospecie®), and can be easily obtained. The first is horseshoe crab lysate that has been fractioned with a Dextran-sulfate Sepharose column and re-composed with the G factor fraction removed. The second is lysate to which carboxymethylated Curdlan (β-D-glycan) has been added in excess to suppress G system activation and make the reagent endotoxin specific. Reagents that are derived from horseshoe crab blood cell components and react specifically with glycans and the like to produce gels are solid by Seikagaku Corp., Maruha and Wako Pure Chemical (β-glycan Test Wako®, etc.). In addition, reagents that produce a gel but are non-specific are available from Seikagaku Corp. (Endotoxin Toxin Test-D®, etc.).

Examples of other desirable gelling reactions include gelling reactions using silkworm body fluid extract, which is activated by glycans such as peptide glycan (one of bacterial cell wall components), β-glycan (a fungal cell wall component) and the like. A silkworm body fluid extract is sold by Wako Pure Chemical as an SLP reagent set (Code #297-51500), and is therefore easy to obtain.

The labeling enzyme used in the present invention may also be an inactive enzyme that has not yet been activated, or a precursor of an active enzyme. Substrate precursors and the like can be used as the substrate of the present invention as well as substrates that are gelled directly by the action of the labeling enzyme. That is, the concept of “substrate” in the present invention includes not only substrates on which the labeling enzyme acts directly, but also precursors of these. Moreover, the substrate used in the present invention may be one kind of substrate or a mixture of two or more kinds. Various activating substances may also be added in addition to the substrate in the present invention. For example, when the gelling reaction is a cascade reaction comprising multiple reactions, an activating substance or the like that activates this cascade reaction can be added to the reaction system.

Method for Detecting and/or Quantifying Target Molecule

Highly sensitive detection of a target molecule can be achieved by using the labeling enzyme of the present invention to amplify a signal obtained from a molecule-recognition reaction using a molecule-recognizing element. The labeling enzyme of the present invention can be used in any molecule-recognition reaction, and therefore the labeling enzyme of the present invention can be used not only in molecule recognition using antibodies, nucleic acids and other biological substances, but also in chemical molecular recognition methods using clathrate compounds, molecular imprinting or the like. Examples of desirable molecular recognition methods of the present invention include molecule recognition methods using such molecule-recognizing elements as antibodies, aptamers and other nucleic acids, proteins, hormone receptors, lecithin, clathrate compounds, physiologically active substance receptors, and template molecules formed by molecular imprinting and the like.

### Enzyme Immunoassay Methods

The present invention can be applied to any known enzyme immunoassay method. Moreover, the present invention can also be applied to immunosensors using enzyme immunoassay methods. The present invention can also be used in methods in which a molecule-recognizing reaction between a molecule-recognizing element and an object of recognition is substituted for the antibody-antigen reaction of an enzyme immunoassay.

In the present invention, an “enzyme immunoassay method” is a method of detecting and/or quantifying a target substance using an enzyme reaction and an immune reaction. More specifically, it is a method in which an antigen-antibody reaction is performed with the antigen or antibody labeled with an enzyme, and the antigen-antibody reaction is detected by measuring enzyme activity to thereby detect and/or quantify a target substance. In general, methods of enzyme immunoassay may be either competitive or non-competitive (such as the sandwich method). Competitive methods are those in which an antigen or antibody is labeled with a labeling enzyme, and the labeled antigen or antibody is made to compete with a free antigen or free antibody in a sample in an antigen-antibody reaction with the corresponding antibody or antigen. A substrate is then added, and the signal from the antigen-antibody reaction is amplified by the enzyme reaction to detect and/or quantify the substance. In the sandwich method, which is the normal non-competitive method, a non-labeled antibody (primary antibody, first antibody) is bound to a target substance (antigen) in a sample, and an enzyme-labeled antibody (secondary antibody, second antibody) is then bound to the complex of the antigen and primary antibody. A substrate is then added, and the signal from the antigen-antibody reaction is amplified by the enzyme reaction to detect and/or quantify the substance. Because the signal is amplified by the secondary antibody, detection sensitivity is particularly high in the sandwich method, and the response range is broad. Other methods of enzyme immunoassay such as the double layer method are also known and the present invention can be applied to these measurement methods. In non-competitive methods such as the sandwich method the epitopes of the primary and secondary antibodies are normally different, but in the present invention they may be the same. When molecule-recognizing elements are used rather than an antigen-antibody reaction, the first molecule-recognizing element and second molecule-recognizing element (corresponding to the primary and secondary antibodies) may have the same recognition site for the target molecule or different recognition sites, but preferably the first and second molecule-recognizing elements have different recognition sites for the target molecule.

### Target Substance

In the present invention, a “target substance” is a substance that is the subject (target) of measurement by the various measurement methods used in the present invention. In the present invention there may also be one or two or more types of target substances. In one embodiment of the present invention, the target substance is specifically recognized by a molecule-recognizing element. Consequently, the target substance of the present invention can be the object of recognition by a molecule-recognizing element. For example, in one embodiment of the present invention if an antibody is adopted as the molecule-recognizing element the target substance is an antigen that is the object of recognition by the molecule-recognizing element.

### Molecule-Recognizing Element

As long as it recognizes the target substance the molecule-recognizing element used in the present invention is not particularly limited and can be selected appropriately according to the object. There are also no particular limits on the mode of molecule recognition, which can be molecule recognition by physical adsorption, chemical adsorption or the like. Consequently, the molecule-recognizing element of the present invention may recognize the target substance by means of hydrogen bonds, intermolecular force (van der Waals force), coordinate binding, ion binding, covalent binding or the like.

As discussed above, specific examples of molecule-recognizing elements of the present invention preferably include antibodies, aptamers and other nucleic acids, proteins, hormone receptors, lecithin, clathrate compounds, physiologically active substance receptors and other biological substances. A template molecule formed by molecular imprinting can also be used as the molecule-recognizing element. Moreover, the object of recognition of these molecule-recognizing elements may be an antigen in the case of an antibody, a nucleic acid, protein, tubulin, chitin or the like in the case of a nucleic acid, a hormone in the case of a hormone receptor, a sugar or the like in the case of a lectin, a component to be clathrated in the case of a clathrate compound, or a physiologically active substance in the case of a physiologically active substance receptor. In the present invention, the idea is that the molecule-recognizing element and the object of recognition are interchangeable. That is, in the case of what is normally thought of as a combination of a molecule-recognizing element and an object of recognition the object of recognition can also be thought of as recognizing the molecule-recognizing element, so the molecule-recognizing element can also be seen as an “object of recognition” and the target of recognition can be seen as a “molecule-recognizing element”. For example in the case of molecule recognition using an antibody and antigen the antibody can be taken as the molecule-recognizing element and the antigen as the object of recognition, or conversely the antibody can be taken as the object of recognition and the antigen as the molecule-recognizing element.

### Antibody

Any antibody capable of producing a specific antigen-antibody reaction with a target antigen (target substance) can be used as the molecule-recognizing element of the present invention without any particular limitations, and either a monoclonal antibody or polyclonal antibody may be used. A monoclonal antibody is preferred because it allows greater sensitivity of detection. When the present invention is applied to ELISA by the sandwich method the epitopes of the primary antibody (first antibody) and secondary antibody (second antibody) may be either the same or different, but preferably they are different. Moreover, a part of an antibody can be used as a molecule-recognizing element in the present invention.

When the antibody recognizing the target substance is a monoclonal antibody for example, it may be induced from mouse ascites using a clone of fused cells obtained by fusing myeloma cells and spleen cells of mice immunized with a purified pathogenic factor, namely a toxin, bacterial cells or the like. A polyclonal antibody may be one purified from serum obtained by immunizing rabbits, goats, rats, sheep, chickens, pigs, donkeys, guinea pigs, dogs, cows or the like with a toxin, bacterial cell or other antigen.

Moreover, a Fab′, Fab or F(ab′)₂ fragment or the like of IgG, IgM, IgE or IgG can be used as the antibody in the present invention. The target antigen (target substance) is also not particularly limited and can be selected appropriately according to the object, but examples include plasma proteins, tumor markers, apoproteins, viruses, auto-antibodies, coagulation and fibrinolytic factors, hormones, drugs in blood, HLA antigens and the like.

Examples of the aforementioned plasma proteins include immunoglobulins (IgG, IgA, IgM, IgD, IgE), complement components (C3, C4, C5, Clq), CRP, α₁-antitrypsin, α₁-microglobulin, β₂-microglobulin, haptoglobin, transferrin, ceruloplasmin, ferritin and the like.

Examples of the aforementioned tumor markers include α-fetoprotein (AFP), carcinoembryonic antigen (CEA), CA19-9, CA125, CA15-3, SCC antigen, prostatic acid phosphatase (PAP), PIVKA-II, γ-seminoprotein, TPA, elastase I, neuron-specific enolase (NSE), immunosuppressive acidic protein (IAP) and the like.

Examples of the aforementioned apoproteins include apo A-I, apo A-II, apo B, apo C-II, apo C-III, apo E and the like.

Examples of the aforementioned viral antigens include hepatitis B virus (HBV) associated antigens, hepatitis C virus (HCV) associated antigens, HTLV-I, HIV and the rabies virus, influenza virus, rubella virus and the like. Examples of such HCV-associated antigens include HCVc100-3 recombinant antigen, pHCV-31 recombinant antigen, pHCV-34 recombinant antigen and the like. Preferably a mixture of these is used. Examples of such HIV-associated antigens include viral surface antigens and the like, such as HIV-I env.gp41 recombinant antigen, HIV-I env.gp120 recombinant antigen, HIV-Igag.p24 recombinant antigen, HIV-II env.p36 recombinant antigen and the like. Examples of non-viral infectious disease antigens include MRSA, ASO, toxoplasma, mycoplasma and STD antigens and the like.

Examples of the aforementioned auto-antibodies include anti-microsome antibodies, anti-thyroglobulin antibodies, antinuclear antibodies, rheumatic factors, anti-mitochondrial antibodies, myelin antibodies and the like.

Examples of the aforementioned coagulation and fibrinolytic factors include fibrinogen, fibrin degradation products (FDP), plasminogen, α₂-plasmin inhibitor, antithrombin III, β-thromboglobulin, factor VIII, protein C, protein S and the like.

Examples of the aforementioned hormones include pituitary hormones (LH, FSH, GH, ACTH, TSH, prolactin), thyroid hormones (T₃, T₄, thyroglobulin), calcitonin, parathyroid hormone (PTH), adrenocortical hormones (aldosterone, cortisol), gonadal hormones (hCG, estrogen, testosterone, hPL), pancreatic and digestive tract hormones (insulin, C-peptide, glucagon, gastrin), and others (renin, angiotensin I and II, enkephalin, erythropoietin).

Examples of the aforementioned drugs in blood include carbamazepine, primidone, valproic acid and other anti-epileptic drugs, digoxin, quinidine, digitoxin, theophylline and other circulatory disease drugs, and gentamicin, kanamycin, streptomycin and other antibiotics and the like.

### Nucleic Acids

A nucleic acid can be used as the molecule-recognizing element of the present invention. A nucleic acid is desirable as the molecule-recognizing element of the present invention from the standpoint of easily forming any sequence, and single-stranded DNA and RNA or the like and double-stranded DNA and RNA and the like can be used. In this case, the mode of molecule recognition by the nucleic acid is not particularly limited, and examples include recognition by double-strand and triple-strand formation. Moreover, a DNA or RNA aptamer with strong affinity for a specific nucleic acid or protein is desirable as the molecule-recognizing element of the present invention because it can be relatively easily obtained using an affinity column or the like. An intercalator can also be used as the molecule-recognizing element of the present invention, and can molecularly recognize a nucleic acid.

### Proteins

Any protein that can recognize a target substance can be used as the molecule-recognizing element of the present invention, without any particular limitations. Consequently, low-molecular-weight (about 6000 to 13,000) proteins and the like having strong affinity for various heavy metals (particularly zinc, cadmium, copper, mercury and the like) can be used as the molecule-recognizing element of the present invention. These have been found in the livers, kidneys and other tissues of animals and recently even in microorganisms, and exhibit an amino acid distribution high in cysteine but with very few aromatic residues.

### Clathrate Compounds

Any clathrate compound with molecule-recognizing ability can be used as the molecule-recognizing element of the present invention without any particular limitations, and can be selected appropriately according to the object but desirable examples include those having tubular (one-dimensional) void cavities, those having layered (two-dimensional) void cavities and those having cage-like (three-dimensional) void cavities.

Examples of clathrate compounds having tubular (one-dimensional) void cavities include urea, thiourea, deoxycholic acid, dinitrodiphenyl, dioxytriphenylmethane, triphenylmethane, methylnaphthalene, spirochromans, PHTP (perhydrotriphenylene), cellulose, amylose, cyclodextrin (with a cage-like void cavity in solution), cyclodextrin derivatives, phenylboric acid and the like. The object of recognition in this case may be a phenol derivative, salicylic acid, a bromic acid derivative or ester, a cholesterol or other steroid, a vitamin such as ascorbic acid, retinol or tocopherol, a hydrocarbon such as limonene, or glucose or the like.

Examples of layered (two-dimensional) clathrate compounds include clay minerals, graphite, smectite, montmorillonite, zeolite and the like. The object of recognition may be a hydrophilic substance, a polar compound, O, HSO₄⁻, a halogen, a halide, an alkaline metal, brucine, codeine, o-phenylenediamine, benzidine, piperidine, adenine, guanine or a liposide of these or H₂O or the like.

Examples of cage-like (three-dimensional) clathrate compounds include hydroquinone, gaseous hydrates, tri-o-thymotide, oxyflavans, dicyanoamminenickel, cryptand, calixarene, crown compounds and the like. Crown compounds here include not only crown ethers having oxygen as an electron-donating donor atom, but also, analogs of these including macrocyclic compounds having nitrogen, sulfur or other donor atoms as ring structure-forming atoms, as well as heterocyclic crown compounds such as cryptand comprising two or more rings. The object of recognition by a cage-like clathrate compound may be chloroform, benzene, toluene, isopropyl alcohol, acetone, methanol or the like for example.

Labeling of Molecule-Recognizing Element with Labeling Enzyme

In the present invention, the molecule-recognizing element is labeled with a labeling enzyme. There are no particular limits on the method of linking the labeling enzyme to the molecule-recognizing compound, which may be a chemical method or a genetic engineering method. Also, the labeling enzyme may be linked either directly or indirectly to the molecule-recognizing element.

When the labeling enzyme is linked to the molecule-recognizing element by chemical means, it can be linked directly as discussed above or may be linked indirectly via a linker or spacer. The specific binding of digoxigenin, avidin, biotin or the like may also be used to link the labeling enzyme to the molecule-recognizing element using. For example, using thrombin as the labeling enzyme and an aptamer as the molecule-recognizing element, the thrombin can be labeled with avidin and the aptamer with biotin, and the two can then be mixed to obtain a thrombin-labeled aptamer by Taq polymerization.

When the labeling enzyme is linked to the molecule-recognizing element by genetic engineering means, the fused protein method can be used for example to prepare an enzyme-labeled antibody or the like as a fused protein (chimera protein).

Specifically, a known method using a bivalent crosslinking agent for example can be used to link the labeling enzyme to the molecule-recognizing element, although this is not a limitation. Therefore, an amino group, carboxyl group, hydroxyl group, thiol group, imidazole group, phenol group or the like may be used. For purposes of binding between amino groups, the isocyanate method, glutaraldehyde method, difluorobenzene method, benzoquinone method or the like may be used for example. For binding an amino group to a carboxyl group, a method of converting the carboxyl group to a succinylimide ester may be used, as well as the carbodiimide method, the Woodward's reagent method, and the periodic acidification method (Nakane method) in which the amino group is crosslinked with a sugar chain. When using thiol groups, the carboxyl group of one side can be converted to a succinylimide ester, and then reacted with cysteine to introduce a thiol group, and the two can then be linked using a thiol-reactive bivalent crosslinking agent. Other methods using phenyl groups include the diazotation method, alkylation method and the like.

When there is no suitable functional group for linking the enzyme to the molecule-recognizing element, an amino group, carboxyl group or thiol group or the like may be introduced. These may be introduced via a spacer to facilitate linking with the enzyme.

The linking ratio of molecule-recognizing element and labeling enzyme is not limited to 1:1 but may be any ratio. For example, multiple labeling enzymes can be linked to a molecule-recognizing element using the glutaraldehyde method or periodic acid method (J. Histochemistry and Cytochemistry 22, 1084, 1974).

### Support

In one embodiment of the present invention, the molecule-recognizing element (antibody or the like) or object of recognition (antigen or the like) can be fixed to a support. For example, when the present invention is applied to the ELISA method, the molecule-recognizing element of the present invention is fixed to a support. Any known material may be used for the support for fixing the molecule-recognizing element or object of recognition of the present invention. Thus, an inorganic polymer compound such as porous glass, silica gel or hydroxyapatite or a metal such as gold, silver or platinum can be used for the support. A synthetic polymer such as ethylene-vinyl acetate copolymer, polyvinyl chloride, polyurethane, polyethylene, polystyrene, nylon, polyester, polycarbonate or the like; a natural polymer such as starch, gluten, chitin, cellulose or a natural gum or the like; or a derivative of one of these may also be used. Other examples of supports in the present invention include agarose derivatives having hydrophobic groups, nitrocellulose, and derivatives of these and the like. In particular, the material of the support can be selected according to the measurement method used in the present invention, and a glass or other highly-reflective support is desirable in the case of measurement by the IER method, while in the case of measurement by the SPR method a thin film of gold or silver is preferred. The support is not particularly limited as to shape, and may be in the form of a microplate, beads, film, sheet, membrane, fiber, stick or the like. A porous substance may also be used for the support.

### Fixing Molecule-Recognizing Element or Object of Recognition to Support

When an antigen or antibody is fixed to a support in the present invention, the fixing method is not particularly limited, and any known method may be used. Thus, physical adsorption, inclusive fixing, or fixing by a chemical linking reaction may be used depending on the molecule-recognizing element, the object of recognition and the support. An example of a physical adsorption method is adsorption of a protein on the surface of a hydrophobic resin. An example of an inclusive fixing method is a method in which fixing is accomplished by including the substance to be fixed in a gel, polymer or other support. An example of a fixing method based on a chemical reaction is a method in which functional groups introduced on the surface of a support are chemically linked to functional groups of the substance to be fixed.

Examples of chemical fixing methods including methods of fixing a molecule-recognizing element or object of recognition to a support using a silane coupling agent, plasma-polymerized film or acid anhydride. For example, when the molecule-recognizing element or object of recognition is fixed to the support by means of covalent bonds using an acid anhydride, the molecule-recognizing element may be fixed via acid anhydride groups present on the surface of the support, or the acid anhydride groups may be introduced into carboxyl, formyl, amino, azide, isocyanate, chloroformyl, epoxy or other reactive function groups present on the surface of the support, after which the molecule-recognizing element or the like can be fixed via these acid anhydride groups. When neither acid anhydride groups nor reactive functional groups exist on the surface of the polymer material, the molecule-recognizing element may be fixed by directly introducing acid anhydride groups into the material surface, or else by first introducing reactive functional groups and then acid anhydride groups. The acid anhydride groups can be introduced by reacting a styrene-maleic anhydride copolymer, ethylene-maleic anhydride copolymer, methyl vinyl ether-maleic anhydride copolymer or the like, or by graft polymerizing maleic anhydride to polyurethane by γ rays or other electron rays.

When introducing carboxyl groups into an ethylene-vinyl acetate copolymer for example, the reactive functional groups can be introduced into the surface of the polymer material by first saponifying and then carboxymethylating the ethylene-vinyl acetate copolymer. The carboxyl groups can also be introduced into azide groups by way of hydrazyl groups. The carboxyl groups can also be converted to chloroformyl groups by chlorination using thionyl chloride, acetyl chloride or the like.

Amino groups can be introduced into ethylene-vinyl acetate copolymer by aminoacetalizing saponified ethylene-vinyl acetate copolymer. Epoxy groups can be introduced by a reaction with epichlorohydrin, diethylene glycol diglycidyl ether or the like. Isocyanate groups can be introduced by a reaction with hexamethylene diisocyanate, tolylene diisocyanate or the like. Formyl groups can be introduced by a reaction with glutaraldehyde, dialdehyde starch or the like.

Various other fixing methods based on chemical linking can also be used, such as introducing various functional groups onto the surface of the support by plasma polymerization using a monomer gas as the raw material. Methods using plasma treatment for example are known for introducing carboxyl, amino and other reactive functional groups into the surface of polymer materials such as polystyrene that lack reactive functional groups. Known methods of plasma treatment include glow discharge treatment, corona discharge treatment and the like using a reactive gas such as oxygen, nitrogen or ammonia, a non-reactive gas such as argon, or a mixed gas such as air. It is particularly desirable to use oxygen when introducing carboxyl groups into a polymer material, and to use ammonia gas when introducing amino groups.

In addition, a molecule-recognizing element can be fixed to a support using a plasma-polymerized film (Tokusaihyo 01/033227). Fixing can also be accomplished by holding oxygen inside an ultrafiltered film and coating the surface with a plasma-polymerized film (Yoshimura, Kikuko et al., “Plasma jugoho ni yoru sensor-yo koso koteikamaku no sakusei”, Bunseki Kagaku, 1990, 39:749-753), or by inclusion using a plasma-polymerized film (Japanese Patents Laid-open No. H06-153971).

Measuring Film Thickness and/or Refractive Index

In the present invention, a target substance can be detected and/or quantified by measuring changes in physical properties (e.g. film thickness, refractive index, density, weight, etc.) accompanying the gelling reaction between the labeling enzyme and its substrate. However, as discussed above, in the method of the present invention the substrate is gelled on the support by the action of the labeling enzyme, a thin film is formed, and the thickness and/or refractive index of the film on the support is altered. Therefore, when using the labeling enzyme of the present invention to detect and/or quantify a target substance, it is preferably the changes in film thickness and/or refractive index that are measured.

Any measuring method capable of measuring changes in film thickness and/or refractive index can be used favorably as the measurement method of the present invention. More specifically, measurement methods that can be used in the present invention include the interference enhanced reflection method (IER), the surface plasmon resonance method (SPR), ellipsometry, and the optical wave-guide method (OWG), but are not limited to these. Optical measurement methods are preferably used in the present invention because they allow for relatively easy and rapid measurement. IER and SPR are more desirable because they employ relatively simple measurement equipment, and IER is still more desirable from the standpoint of flexibility and easy development of flexible equipment.

Moreover, changes in film thickness and/or refractive index can be measured either continuously or intermittently in the present invention. They can also be measured in real time. Not only can the target substance be detected in the present invention, but it can also be quantified by preparing a calibration curve or the like.

### Interference Enhanced Reflection Method (IER)

IER is a method using interference between light introduced at a particular angle and reflected from two different surfaces, the air side (or solution side) and the support side of the film. For example, at a particular film thickness the strength of the reflected light increases as the film thickness increases because the phases of the light from the two surfaces reinforce one another, while at a different film thickness the strength of the reflected light decreases as the film thickness increases because the phases of the light from the two surfaces attenuate each other. At the film thickness at which the reflective strength starts to strengthen instead of weakening, an anti-reflective coating (AR coating) is produced with a reflective strength of zero. Consequently, in IER the detected strength of the reflected light becomes a sine curve according to the optical thickness (refractive index times film thickness) of the film. Thus, when the film thickness and/or refractive index of the film is altered due to the effect of the labeling enzyme of the present invention, such changes in film thickness and/or refractive index can be measured by IER to detect the target substance. IER methods are described in Japanese Patents Laid-open Nos. H06-222006, H07-260773, H06-341894, H08-184560, H09-329553, H10-104163 and 2004-117325, etc.

### Surface Plasmon Resonance (SPR) Method

SPR is a method of measurement using the fact that when light of a specific wavelength is directed at a specific angle at a thin-film metal surface, the energy of the photons is adsorbed by the electrons of the metal surface and is not reflected. In actual measurement, gold, silver or platinum is often used as the metal, and normally the molecule-recognizing element is linked to a gold surface using a chip comprising a thin gold film attached to a glass or other support. Consequently, mass fluctuations near the metal surface due to changes in the film thickness can be measured by SPR in the present invention. SPR methods are described for example in Japanese patent Laid-open Nos. H09-96605 and H11-183372, etc.

### Ellipsometry

Ellipsometry (also called polarization analysis) is a measurement method in which changes in light polarization are measured and used to calculate the thickness, refractive index and the like of a film.

### Optical Wave-guide (OWG) Method

The OWG method is a method of measurement using evanescent waves generated by light passing through an optical wave-guide, using the fact that the evanescent waves are absorbed and the transmitted light decreases when there is a sample on the surface of the film. OWG methods are described in Japanese Patent Applications Laid-open Nos. H08-114547 and H10-33801 for example.

### Target Substance and Sample

The target substance that can be detected and/or quantified by the method of the present invention is not particularly limited and can be any substance that can be recognized by the molecule-recognizing element of the present invention as described above. Thus, endocrine disruptors, agricultural chemicals, various drugs, hormonally active agents, hormones and nucleic acids as well as viruses, bacteria and other microorganisms can be detected and/or quantified by the present invention. More specifically, examples of endocrine disruptors include dioxins, bisphenol A, alkylphenols, benzopyrenes, PCBs, phthalic acid esters and the like, but are not limited to these. Examples of agricultural chemicals include amitrole, simazine, parathion, benomyl and the like but are not limited to these.

Consequently, the fields of use of the present invention include a wide range of uses such as clinical, pharmaceutical, food and environmental analysis, and the present invention can be used to specify the causative substance in atopic dermatitis or to measure the scattered amount of cedar pollen for example.

A variety of samples can also be analyzed with the present invention. Therefore, in addition to soil, river water, tap and sewer water, air and other environmental samples, blood (whole blood, plasma, serum), lymph, saliva, urine, tissue samples and other biological samples can also be analyzed by the present invention. In the case of pre-natal testing and the like, fetal cells in amniotic fluid or part of dividing egg cells in a test tube can also be used as specimens. The sample to be analyzed in the present invention can of course be treated or pre-treated as necessary. For example, such specimens can be treated to disrupt the cells by enzyme treatment, heat treatment, surfactant treatment, ultrasound or a combination of these for example, either directly or after having been concentrated as a precipitate by centrifugation or the like. Consequently, if the target substance is an organic compound such as a dioxin it can be extracted with an organic solvent.

### BRIEF DESCRIPTION OF THE DRAWINGS

FIG. 1 shows changes in an IER signal when fibrinogen was added to a support having thrombin fixed thereon (Example 1). In the figure, (1) indicates that fibrinogen was added to a support with thrombin fixed thereon, while (2) indicates that fibrinogen was added to a support with BSA fixed thereon.

FIG. 2 shows changes in an IER signal when a thrombin-labeled HSA antibody was added to a support with HSA fixed thereon (Example 3). In the figure, (1) indicates that the thrombin-labeled HSA antibody was added to a support with fixed HSA, while (2) indicates that the thrombin-labeled HSA antibody and HSA were added to the support with fixed HSA, and (3) indicates that the thrombin-labeled HSA antibody was added to a support with BSA fixed thereon.

FIG. 3 shows the IER signal when a thrombin-labeled HSA antibody and HSA were added to a support with HSA fixed thereon (Example 3-2).

FIG. 4 shows the IER signal when a thrombin-labeled HSA antibody and BSA were added to a support with HSA fixed thereon (Example 3-2).

FIG. 5 shows changes in the IER signal on the vertical axis and the added amount of HSA or BSA on the horizontal axis based on FIGS. 3 and 4 (Example 3-2).

FIG. 6 shows the IER signal measured without a washing step (Example 4).

### BEST MODE FOR CARRYING OUT THE INVENTION

### Labeling Enzyme

In one embodiment of the present invention, the invention is a labeling enzyme. This labeling enzyme of the present invention is an enzyme that catalyzes a substrate-gelling reaction and can be used in various kinds of labeling as described above. Examples include proteases, peptides and the like capable of catalyzing such a gelling reaction (in which case the substrate may be a protein, polypeptide or the like). Of these, an enzyme participating in a blood coagulation reaction is desirable, and thrombin is particularly desirable (in which case the substrate in fibrinogen).

Method for Detecting and/or Quantifying Target Substance

In one embodiment of the present invention, the invention is a method of detecting and/or quantifying a target substance by enzyme immunoassay. Thus, when the present invention is used in the sandwich method for example it may comprise the following steps:

(a) a step of reacting a sample comprising a target substance, a fixed antibody comprising a support having fixed thereon an antibody (primary antibody) capable of specifically recognizing the target substance, and a labeled antibody (secondary antibody) capable of specifically recognizing the target substance and labeled with a labeling enzyme that catalyzes a reaction of gelling a substrate;

(b) a step of adding a substrate that is gelled by the catalytic action of the labeling enzyme to the fixed antibody/target substance/labeled antibody complex produced in step (a), thereby producing a gel on a support; and

(c) a step of measuring changes in the film thickness and/or refractive index of a film on the support resulting from the gel produced in step (b).

Moreover, the competitive immunoassay method of the present invention comprises the following steps for example:

(a) a step of reacting a sample comprising a target substance, a sample comprising an antigen labeled with a labeling enzyme that catalyzes a reaction of gelling a substrate, and a fixed antibody comprising a support having fixed thereon and an antibody capable of specifically recognizing the target substance and the antigen;

(b) a step of adding a substrate that is gelled by the catalytic action of the labeling enzyme to the fixed antibody-target substance complex and fixed antibody-labeled antigen produced in step (a), thereby producing a gel on the support; and

(c) a step of measuring changes in the film thickness and/or refractive index of a film on the support resulting from the gel produced in step (b).

In this embodiment, the target substance and the antigen labeled with the labeling enzyme capable of catalyzing the substrate-gelling reaction may be contained in the same sample.

### Measurement Kit

One embodiment of the present invention provides a target substance measurement kit. In one embodiment, the target substance measurement kit of the present invention comprises a labeling enzyme that catalyzes a substrate-gelling reaction, a substrate that is gelled by the catalytic action of this labeling enzyme, and a molecule-recognizing element capable of specifically recognizing the target substance. The measurement kit of the present invention may also contain a support for fixing a molecule-recognizing element or object of recognition (such as an antibody or antigen), a standard solution, a gelling reaction amplifier, a buffer, instructions for use, a package or the like.

One embodiment of the present invention provides a kit for labeling a molecule-recognizing element. In one embodiment, the kit of the present invention comprises a labeling enzyme that catalyzes a substrate-gelling reaction, and a reagent for linking this labeling enzyme to a molecule-recognizing element. There may be one or two or more linking reagents. This kit may also include other components, such as for example, a solution (such as a buffer) for preserving the molecule-recognizing element labeled by the labeling enzyme, a support for fixing the molecule-recognizing element or object of recognition (such as an antibody or antigen), a standard solution, a gelling reaction amplifier, a buffer, instructions for use, a package or the like.

### Biosensor

One embodiment of the present invention provides a biosensor for detecting and/or quantifying a target substance.

In one embodiment, the biosensor of the present invention comprises: (a) a reaction part for reacting a first molecule-recognizing element (such as a primary antibody) fixed on a support and capable of specifically recognizing the target substance, a second molecule-recognizing element (such as a secondary antibody) capable of specifically recognizing the target substance and labeled with a labeling enzyme that catalyzes a substrate gelling reaction, a substrate that is gelled by the catalytic action of the labeling enzyme, and a target substance, and (b) a measurement part for measuring changes in the film thickness and/or refractive index of a film on a support resulting from the gel produced by this reaction.

Moreover, in one embodiment the biosensor of the present invention comprises: (a) a reaction part for reacting a target substance, a target substance labeled with a labeling enzyme that catalyzes a substrate-gelling reaction, a fixed molecule-recognizing element fixed on a support and capable of specifically recognizing the target substance, and a substrate that is gelled by the catalytic action of the labeling enzyme, and (b) a measurement part for measuring changes in the film thickness and/or refractive index of a film on a support resulting from the gel produced by this reaction.

### EFFECTS OF THE INVENTION

The present invention allows a target substance to be detected and/or quantified with high sensitivity and with little effect from coexisting substances. The present invention also allows a target substance to be detected and/or quantified with simple equipment.

### EXAMPLES

The present invention is explained in detail below using examples, but the present invention is not limited to these examples.

### Example 1

A gel produced by thrombin and fibrinogen was measured by IER.

### Experimental Procedures

1. A glass plate (14.5 mm×14.5 mm) was incubated overnight at 99° C. in 10% 3-aminopropyltriethoxysilane (γ-APTES: Shinetsu Chemical), and then treated for 3 hours at room temperature with 2% glutaraldehyde solution (Wako Pure Chemical).
2. 1 ml of thrombin solution (human thrombin 10 μg/ml: Wako Pure Chemical) was added on the same plate, and incubated for 3 hours at room temperature to fix the thrombin on the plate.
3. The plate was washed, fibrinogen solution (2 mg/ml, total volume 200 μl: Wako Pure Chemical) was added, and the IER signal was measured.
4. A plate having bovine serum albumin (BSA: Pierce) fixed thereon was prepared by the same procedures, and the IER signal was measured.

### Measurement Results

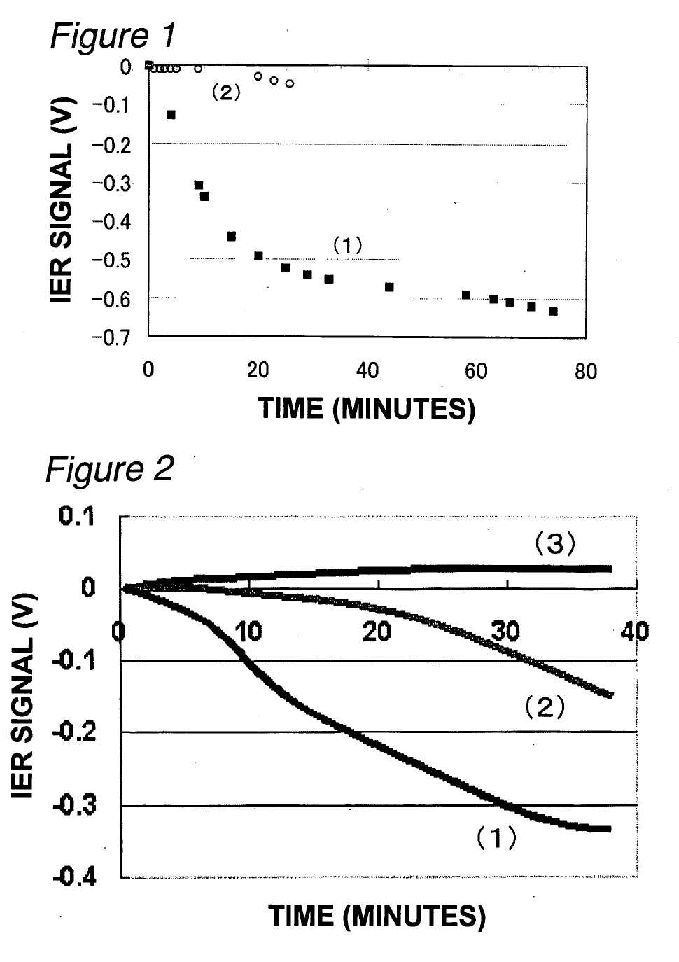

The results are shown in FIG. 1. When fibrinogen was added to the plate with thrombin fixed thereon, the fibrinogen gelled, and it was shown that changes in film thickness and/or refractive index due to this gel could be measured by IER. When fibrinogen was added without fixing the thrombin on the plate, no great change in IER signal was measured. That is, a large change in IER signal was only measured when a thin film was formed very near the plate. In the case of the plate with BSA fixed thereon, no change in IER signal was measured. It is thought that in this case no change in IER signal was measured because the BSA did not cause the fibrinogen to gel.

This confirms that when fibrinogen as the substrate is added to a plate having thrombin fixed thereon as the labeling enzyme, the fibrinogen is broken down into fibrin to form a gel, and changes in film thickness and/or refractive index due to this gelling can be measured by IER.

### Example 1-2

A gel produced by thrombin and fibrinogen was measured by ellipsometry.

### Experimental Procedures

Gelling caused by the reaction of thrombin and fibrinogen was measured as in Example 1 except that the plate with the thrombin fixed thereon was measured by ellipsometry.

The ellipsometry measurement conditions were as follows.

Automatic ellipsometer: DHA-XA (Mizojiri Optical)

Light source: He—Ne laser, wavelength 632.8 nm

Incidence angle: 70 degrees

### Measurement Results

A gel produced by the reaction of thrombin and fibrinogen was confirmed from the measurement results, with a gel thickness of 113.5 nm.

This confirms that when fibrinogen as the substrate is added to a plate having thrombin fixed thereon as the labeling enzyme, the fibrinogen is broken down into fibrin to form a gel, and changes in film thickness and/or refractive index due to this gelling can be measured by ellipsometry. Moreover, it was confirmed from measurement of gel film thickness by ellipsometry that the gel produced by fibrinogen and thrombin fixed on a plate occurred near the plate.

### Example 2

### Experimental Procedures

1. Glass plates (14.5 mm×14.5 mm) were incubated overnight at 99° C. in 10% 3-aminopropyltriethoxysilane (γ-APTES: Shinetsu Chemical), and then treated for 3 hours at room temperature with 2% glutaraldehyde solution (Wako Pure Chemical).
2. Thrombin solutions of differing concentrations (bovine thrombin 10 μg/ml and 1 μg/ml: Wako Pure Chemical) were each added to a plate in the amount of 1 ml, and incubated for 3 hours at room temperature to fix the thrombin on the plate.
3. The plates were washed, fibrinogen solution (2 mg/ml, total volume 200 μl: Wako Pure Chemical) was added, and the IER signal was measured.
4. A plate having bovine serum albumin (BSA: Pierce) fixed thereon was prepared by the same procedures, and the IER signal was measured.

### Measurement Results

The results are shown in Table 1. When 10 μg/ml and 1 μg/ml bovine thrombin solutions were added to plates, changes in IER signal were confirmed 8 minutes after addition of fibrinogen to the plates. When BSA was added to the plate, little change in IER signal was detected. These results confirm that when fibrinogen is added to a plate having thrombin fixed thereon, a gel is produced, and changes in the thickness and/or refractive index of the resulting film can be measured by IER.

TABLE 1

| | ΔIER signal (V) | | |
|---|---|---|---|
| | Added amount of | | Added amount |
| | bovine thrombin | | of BSA |
| Time (min) | 10 μg/ml | 1 μg/ml | 10 μg/ml |
| 0 | 0.00 | 0.00 | 0.00 |
| 1 | −0.06 | −0.08 | 0.00 |
| 2 | −0.07 | −0.10 | 0.00 |
| 3 | −0.07 | −0.10 | −0.01 |
| 4 | −0.09 | −0.09 | −0.01 |
| 5 | −0.12 | −0.11 | −0.01 |
| 6 | −0.17 | −0.11 | −0.02 |
| 7 | −0.23 | −0.11 | −0.02 |
| 8 | −0.20 | −0.11 | −0.02 |

### Example 3

### Experimental Procedures

A. Thrombin labeling of antibody

An antibody was thrombin labeled by the maleimide non-hinge method according to the following procedures.

1. Introduction of SH groups into IgG

(1) A 50 mM potassium phosphate buffer was prepared containing 150 mM NaCl and 1 mM EDTA, and adjusted to a pH of 7.0 (hereunder, buffer A).

(2) 0.5 mg of IgG was added to 500 μl of buffer A to prepare an IgG solution.

(3) A DMSO solution (20 mM, 80 μl) of N-succinimidyl-3-(2-pyridyldithio) propionate (SPDP, Pierce) was added to this IgG solution, and incubated for 10 minutes at 30° C. to form disulfide bonds.

(4) This was dialyzed with buffer A to remove unreacted SPDP.

(5) 12 mg of dithiothreitol (DTT, Wako Pure Chemical) was added to 1 ml of IgG sample, and incubated for 30 minutes at room temperature to introduce SH groups.

(6) Following incubation this was immediately cooled and dialyzed with PBS to remove the DTT.

2. Introduction of maleimide groups into thrombin

(1) A 50 mM calcium phosphate buffer was adjusted to a pH of 7.0 (hereunder, buffer B).

(2) 400 μg of thrombin was added to 1 ml of buffer B to prepare a thrombin solution.

(3) 250 μg of Sulfo-SMCC (Pierce) was added to this thrombin solution, and incubated for 10 minutes at 30° C.

(4) Following incubation, this was immediately cooled.

(5) This was treated with a NAP10 desalting column, and then with phosphate buffer (PBS).

3. Linking of thrombin to IgG

The IgG with introduced SH groups prepared in 1 above was mixed with the thrombin with introduced maleimide groups prepared in 2, and incubated for 4 hours or more at 4° C.

B. Detection of HSA with thrombin-labeled antibody

(1) HSA (human serum albumin) and BSA were fixed on glass plates as in Example 1.

(2) 200 μl of solution containing about 400 μg/ml of thrombin-labeled antibody was added to an HSA-fixed plate and incubated at room temperature for 30 minutes, after which the antibody solution was removed and the plate was washed 3 times in PBS. 200 μl of a 2 mg/ml fibrinogen solution was then added, and the IER signal was measured.

(3) 3.5 mg of HSA (about 100 times the molar amount of the antibody) was added to 200 μg of thrombin-labeled antibody solution, and incubated at room temperature for 10 minutes. 200 μl of this incubated solution was then added to an HSA-fixed plate and incubated for 30 minutes at room temperature, after which the antibody solution was removed and the plate was washed 3 times in PBS. 200 μl of a 2 mg/ml fibrinogen solution was then added, and the IER signal was measured.

(4) The IER signal was measured as in (2) above using a BSA-fixed plate.

### Measurement Results

The results are shown in FIG. 2. When a thrombin-labeled HSA antibody was added to an HSA-fixed plate (1), a large change in IER signal was measured, and it was possible to detect changes in the thickness and the like of the film on the HSA-fixed plate due to the gelling reaction of thrombin and fibrinogen. On the other hand, when a previously mixed solution of thrombin-labeled HSA antibody and HSA was added to an HSA-fixed plate (2), the IER signal did not change as much as in (1). That is, there was a significant difference in the change in the IER signal depending on whether (1) only a thrombin-labeled HSA antibody or (2) a thrombin-labeled HSA antibody and competing HSA were added to a plate having HSA fixed thereon. There was almost no change in IER signal when a thrombin-labeled HSA antibody was added to a BSA-fixed plate (3), confirming that in this case there was almost no change in the film thickness and/or refractive index. These results indicate that HSA in a sample can be detected by the method of the present invention.

### Example 3-2

### Experimental Procedures

An antibody was labeled according to the methods described in “A. Thrombin labeling of antibody” in Example 3, and the gel occurring on the substrate was measured by IER using methods similar to those described under “B. Detection of HSA with thrombin-labeled antibody”. The assay was by the same competitive method used in Example 3, with 500 pM, 5 nM, 50 nM, 500 nM and 5 μM of HSA added to the HSA-fixed plate in addition to the thrombin-labeled antibody. 500 pM, 5 nM, 50 nM, 500 nM and 5 μM of BSA was also added to HSA-fixed plates as a control instead of the thrombin-labeled antibody.

### Measurement Results

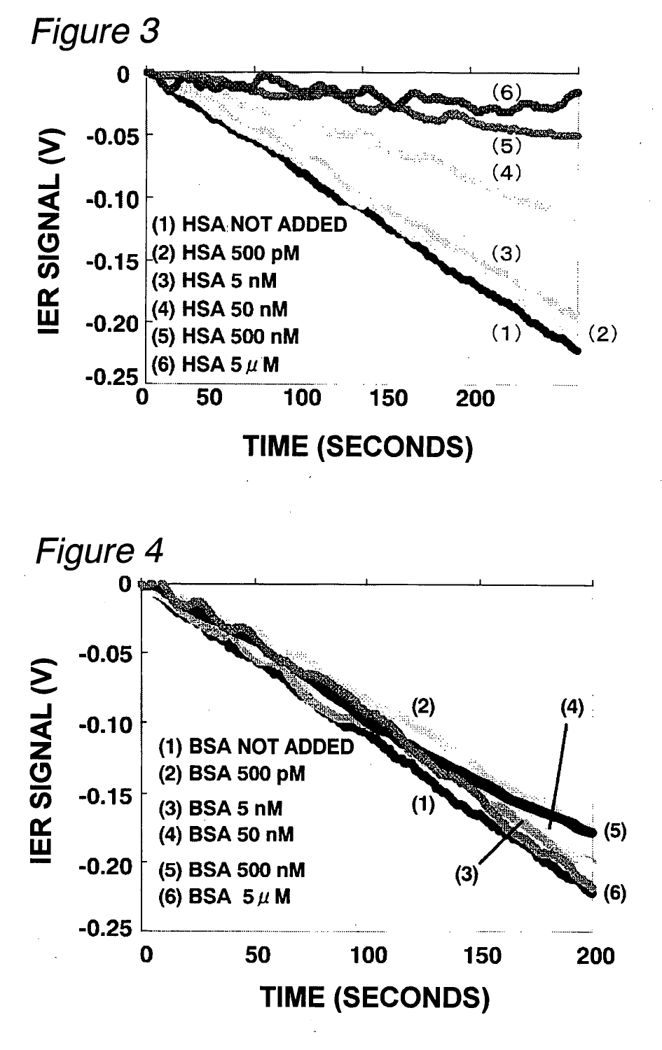

As shown in FIG. 3, when no HSA was added in addition to the thrombin-labeled HSA antibody, the reaction between the thrombin-labeled HSA antibody and the HSA on the plate was not competitively inhibited, and so there was considerable binding between the thrombin-labeled HSA antibody and the HSA on the plate, and a gel was produced on the plate when fibrinogen was added, resulting in a large change in IER signal. When HSA was added in addition to the thrombin-labeled HSA antibody, on the other hand, the reaction between the thrombin-labeled HSA antibody and the HSA on the plate was inhibited by the added HSA, so that the thrombin-labeled HSA antibody reacted less with the HSA on the plate, and little gel was produced even when the fibrinogen was lowered, resulting in a smaller change in the IER signal.

The results for BSA are shown in FIG. 4. Because BSA does not inhibit the reaction between the thrombin-labeled HSA antibody and the HSA on the plate, the IER signal was the same even when the concentration of added BSA was changed.

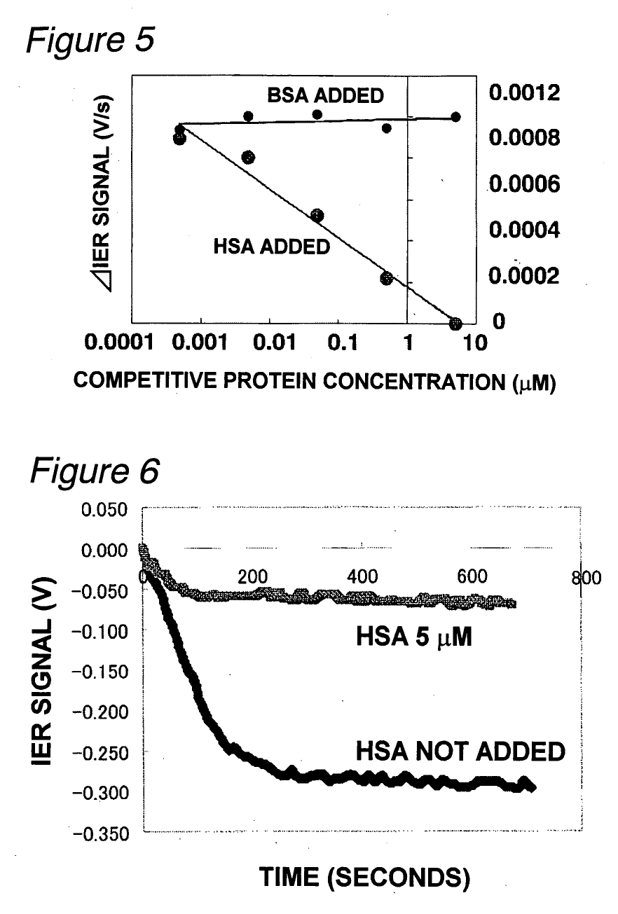

In FIG. 5, the change in IER signal is shown on the vertical axis and the added amount of HSA or BSA on the horizontal axis. It is thus shown that even at an HSA concentration of about 5 nM, adequate detection can be achieved with the method of the present invention.

### Example 4

### Experimental Procedures

An antibody was thrombin labeled according to the methods described under “A. Thrombin labeling of antibody” in Example 3. The gel occurring on the plate was then measured by IER by methods similar to those described under “B. Detection of HSA with thrombin-labeled antibody” in Example 3, except that the washing step was omitted. The assay was by the same competitive method used in Example 3, and the IER signal was measured when 5 μM of HSA was added to the HSA-fixed plate in addition to the thrombin-labeled antibody, and when only the thrombin-labeled antibody was added, with no HSA added to the HSA-fixed plate.

### Measurement Results

As shown in FIG. 6, when no HSA was added in addition to the thrombin-labeled HSA antibody there was a large change in IER signal (ΔIER signal=−0.00181 V/sec), while when HSA was added in addition to the thrombin-labeled HSA antibody, there was little change in IER signal (ΔIER signal=−0.00058 V/sec).

It was thus shown that a target substance can be measured with high sensitivity in the present invention even without a washing step, which is required in conventional methods. It is believed that in the method of the present invention the target substance could be measured without any particular washing step because the gel is measured near the plate, but the present invention is not restricted by this theory.

1. A labeling enzyme that catalyzes a reaction of gelling a substrate.

2. The labeling enzyme according to claim 1, wherein the labeling enzyme is a protease or peptidase.

3. The labeling enzyme according to claim 1, wherein the labeling enzyme is an enzyme involved in a blood coagulation reaction.

4. The labeling enzyme according to claim 1, wherein the labeling enzyme is thrombin.

5. The labeling enzyme according to claim 1, wherein the substrate is a protein.

6. The labeling enzyme according to claim 1, wherein the substrate is fibrinogen.

7. A method for detecting and/or quantifying a target substance, comprising:
(a) reacting a target substance, a fixed molecule-recognizing element comprising a support having fixed thereon a molecule-recognizing element capable of specifically recognizing the target substance, and a labeled molecule-recognizing element capable of specifically recognizing the target substance and labeled with a labeling enzyme that catalyzes a reaction of gelling a substrate;
(b) adding a substrate that is gelled by the catalytic action of the labeling enzyme to the fixed molecule-recognizing element/target substance/labeled molecule-recognizing element complex produced in (a), thereby producing a gel on the support; and
(c) measuring changes in the film thickness and/or refractive index of a film on the support comprising the gel produced in (b).

8. A method for detecting and/or quantifying a target substance, comprising:
(a) reacting a target substance and a target substance labeled with a labeling enzyme that catalyzes a substrate-gelling reaction, with a fixed molecule-recognizing element comprising a support having fixed thereon a molecule-recognizing element capable of specifically recognizing the target substance;
(b) adding a substrate that is gelled by the catalytic action of the labeling enzyme to the fixed molecule-recognizing element/target substance complex and the fixed molecule-recognizing element/labeled target substance produced in (a), thereby producing a gel on the support; and
(c) measuring changes in the film thickness and/or refractive index of a film on the support comprising the gel produced in (b).

9. The method for detecting and/or quantifying a target substance according to claim 7, wherein changes in the film thickness and/or refractive index of the film are measured by the interference enhanced reflection method or surface plasmon resonance method.

10. A kit for measuring a target substance, comprising:
a labeling enzyme that catalyzes a reaction of gelling a substrate; and
a substrate that is gelled by the catalytic action of the labeling enzyme.

11. A kit for measuring a target substance, comprising:
a labeling enzyme that catalyzes a reaction of gelling a substrate; and
a reagent for linking the labeling enzyme to a molecule-recognizing element capable of specifically recognizing a target substance.

12. A biosensor for detecting and/or quantifying a target substance, comprising:
(a) a reaction part for reacting a first molecule-recognizing element fixed on a support and capable of specifically recognizing a target substance, a second molecule-recognizing element labeled with a labeling enzyme that catalyzes a reaction of gelling a substrate and capable of specifically recognizing the target substance, a substrate that is gelled by the catalytic action of the labeling enzyme, and the target substance; and
(b) a measurement part for measuring changes in the film thickness and/or refractive index of a film on the support due to the gel produced by said reaction.

13. A biosensor for detecting and/or quantifying a target substance, comprising:
(a) a reaction part for reacting a target substance, a labeled substance labeled with a labeling enzyme that catalyzes a reaction of gelling a substrate, a fixed molecule-recognizing element fixed on a support and capable of specifically recognizing the target substance, and a substrate that is gelled by the catalytic action of the labeling enzyme; and
(b) a measurement part for measuring changes in the film thickness and/or refractive index of a film on the support due to the gel produced by said reaction.
